(19) United States
(12) Patent Application Publication
(10) Pub. No.: US 2013/0018416 A1
(43) Pub. Date: Jan. 17, 2013

(54) SOFT SUTURE ANCHOR MADE OF BRAIDED OR MONOFILAMENT SUTURE

(71) Applicants: Giuseppe LOMBARDO, New Port Richey, FL (US); Steven E. FITTS, Largo, FL (US); Jean-Paul DEEB, Land O' Lakes, FL (US); Peter C. MILLER, Largo, FL (US)

(72) Inventors: Giuseppe LOMBARDO, New Port Richey, FL (US); Steven E. FITTS, Largo, FL (US); Jean-Paul DEEB, Land O' Lakes, FL (US); Peter C. MILLER, Largo, FL (US)

(73) Assignee: LINVATEC CORPORATION, Largo, FL (US)

(21) Appl. No.: 13/448,324

(22) Filed: Apr. 16, 2012

Related U.S. Application Data
(60) Provisional application No. 61/517,230, filed on Apr. 15, 2011.
(60) Provisional application No. 61/517,221, filed on Apr. 15, 2011.
(60) Provisional application No. 61/517,203, filed on Apr. 15, 2011.

(51) Int. Cl. A61B 17/06

(52) U.S. Cl. 606/232

(57) ABSTRACT

A soft suture anchor is described that includes a first section of suture bent to define a first vertex separating a first standing end and a second standing end. The first standing end and the second standing end being legs of an acute angle. A second section of suture circumscribes the first standing end and the second standing end with at least one wrap. A first end of said second section is wrapped a plurality of times about the first standing end, and a second end of the second section is wrapped a plurality of times about the second standing end.

### CROSS REFERENCE TO RELATED APPLICATIONS

This application is a non-provisional of and claims the benefit of priority from the following U.S. Provisional Patent Applications: (i) Ser. No. ZL61/517,230, entitled “Suture anchor made from one continuous filament”, and filed on Apr. 15, 2011; (ii) Ser. No. 61/517,221, entitled “Filament suture anchor formed with driver”, and filed on Apr. 15, 2011; (iii) Ser. No. 61/517,203, entitled “Soft distendable suture anchor”, and filed on Apr. 15, 2011. The content of each of the above-identified applications is incorporated herein by reference in its entirety.

### FIELD OF THE INVENTION

The present invention is related to a suture anchor made entirely of braided or monofilament suture.

### BACKGROUND OF THE INVENTION

Suture anchors are commonly employed during surgical procedures to provide a reliable attachment location for sutures in or against a substrate, those attached sutures then being used to capture and retain other objects, such as soft tissue. As such, the suture anchor plays an important role in attaching objects, such as soft tissue to a substrate. The substrate may be bony tissue or soft tissue. In the case of bony tissue, suture anchors are generally inserted into a pre-formed hole in the bone, so that suture extends out of the hole from the anchor. In the case of soft tissue, suture anchors generally are placed on a side of the soft tissue such that suture extends through a hole in the tissue to extend beyond the soft tissue on a side opposite the anchor.

Commonly, such suture anchors contain at least one ridged member which deforms to create an interference fit with a substrate, the interference fit creating a retention capacity of the anchor. Other suture anchors contain some external feature, such as barbs or screw threads, which interacts with the substrate, through piercing, cutting and/or deforming the substrate, to create a retention capacity. Other anchors include multiple features, such as deployable barbs, to create retention capacity.

Soft suture anchors have also been developed, such as the Biomet JuggerKnot™ (a trademark of Biomet Corporation), which utilizes a stiff braded line, which appears to function as a barb against the side of a hole in a substrate.

Many factors have a direct effect on the actual retention capacity achieved by any suture anchor. For example, the quality of tissue, bony or soft, may increase or decrease the retention capacity by a large degree depending on the design of a particular suture anchor. Similarly, the quality of installation affects the retention capacity. As evidenced by the large number of suture anchors on the market, some suture anchors perform in certain circumstances while other anchors perform better in other circumstances.

In light of the forgoing, there continues to be a need for a suture anchor that can provide a relatively more reliable retention capacity in a variety of substrates and when installed under a variety of complex conditions.

### SUMMARY OF THE INVENTION

In accordance with various embodiments of the present invention, a soft suture anchor is described that can reliably provide an increased diameter in a deployed condition for the creation of retention capacity. Further in accordance with various embodiments of the present invention a method of manufacturing is provided for the creation of such soft suture anchors.

### BRIEF DESCRIPTION OF THE DRAWINGS

So that the manner in which the above recited features of the present invention can be understood in detail, a more particular description of the invention briefly summarized above may be had by reference to the figures, some of which are illustrated and described in the accompanying appendix. It is to be noted, however, that the appended documents illustrate only typical embodiments of this invention and are therefore not to be considered limiting of its scope, for the invention may admit to other equally effective embodiments. Moreover, the drawings are not necessarily to scale, with emphasis generally being placed upon illustrating the principles of certain embodiments of invention.

Thus, for further understanding of the nature and objects of the invention, references can be made to the following detailed description, read in connection with the specification following below in which:

FIG. 1 represents an initial step for manufacturing a soft suture anchor in accordance with a first embodiment of the present invention;

FIG. 2 represents a subsequent step for manufacturing the first embodiment of the present invention;

FIG. 3 represents a subsequent step for manufacturing the first embodiment of the present invention;

FIG. 4 represents a subsequent step for manufacturing the first embodiment of the present invention;

FIG. 5a represents a subsequent step for manufacturing the first embodiment of the present invention;

FIG. 5b represents a subsequent step for manufacturing the first embodiment of the present invention;

FIG. 6 represents a subsequent step for manufacturing the first embodiment of the present invention;

FIG. 7 represents an initial step for manufacturing a soft suture anchor in accordance with a second embodiment of the present invention;

FIG. 8 represents a subsequent step for manufacturing the second embodiment of the present invention;

FIG. 9 represents a subsequent step for manufacturing the second embodiment of the present invention;

FIG. 10 represents a subsequent step for manufacturing the second embodiment of the present invention;

FIG. 11 represents a subsequent step for manufacturing the second embodiment of the present invention;

FIG. 12 represents a subsequent step for manufacturing the second embodiment of the present invention;

FIG. 13 represents an embodiment of the present invention;

FIG. 14 represents an enlarged view of the embodiment represented in FIG. 13;

FIG. 15 represents a third embodiment of the present invention;

FIG. 16 represents first step in accordance with a fourth embodiment of the present invention;

FIG. 17 represents second step in accordance with the fourth embodiment of the present invention;

FIG. 18 represents a third step in accordance with the fourth embodiment of the present invention;

FIG. 19 represents a fourth step in accordance with the fourth embodiment of the present invention;

FIG. 20 represents a fifth step in accordance with the fourth embodiment of the present invention;

FIG. 21 represents a sixth step in accordance with the fourth embodiment of the present invention;

FIG. 22 represents a seventh step in accordance with the fourth embodiment of the present invention;

FIG. 22 represents a suture anchor made in accordance with the fourth embodiment of the present invention; and

FIGS. 23a and 23b represent a fifth embodiment of the present invention.

### DETAILED DESCRIPTION OF THE INVENTION

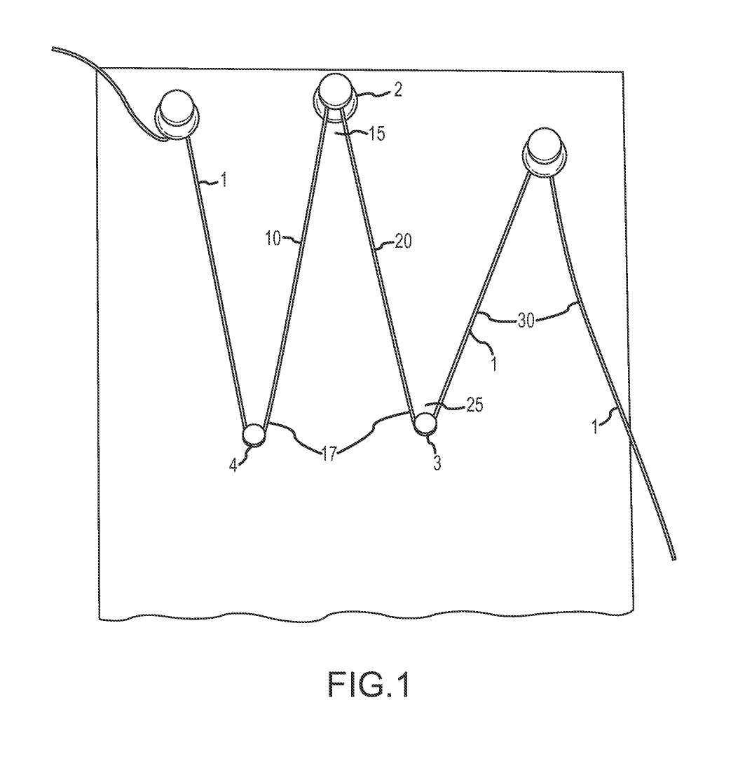

With reference to FIGS. 1-15, there is provided a soft suture anchor, and method for manufacturing the same, embodiments of which are configured so that in a deployed state, the soft suture anchor enlarges to a predictable, enlarged diameter. This enlarged diameter may be used to laterally displace cancellous tissue forming the sides of a preformed bone hole and to engage the harder cortical layer of the bony tissue. This enlarged diameter may also be used to engage an outer surface of a bony or soft tissue to prevent passage of the enlarged diameter through an adjacent hole in that bony or soft tissue.

Before discussing specific embodiments, it may be helpful to understand that each of the suture anchors discussed herein contain two sections: a “First Section” of suture, which is the suture anchored at one end and having two standing ends extending from the anchor to be later used by a surgeon; and a “Second Section” of suture, which is to form a portion of the anchor that increases in diameter as part of deployment. Even though it is the Second Section that increases in diameter at deployment, it should be understood that the First Section of suture also play a role in the anchor even though the First Section remains free to slide in relation to the Second Section. The First Section of suture helps to position, align and support the Second Section of suture, such that if the First Section of suture were to be removed from the Second Section of suture after deployment of the anchor, the Second Section may be free to spill (i.e., release), allowing the Second Section to collapse and shrink in size, allowing for easy removal.

In other words, the Second Section has two primary functions. First, it becomes a base for the First Section to slide within. Second, when compressed during deployment, the Second Section becomes more compact in one direction thereby expanding outwardly and increasing its overall diameter to create a retention capacity. This action of having the Second Section change in shape to increase its overall diameter is a useful characteristic which may be used advantageously to secure the suture anchor into a hole in or against a bony or soft tissue. It is this combination of the expanding Second Section coupled with the First Section slidable in relation to the Second Section that render the present invention ideal for the reattachment of soft tissue to bone or soft tissue to soft tissue where it is desirable to pass sliding knots to secure a repair.

The term “Standing End” is used throughout the following to refer to one or both of the ends of a First Section that will ultimately be placed under load by a surgeon during surgery. The two standing ends are separated from one another by a Bight (i.e., any curved section between two Standing Ends). In the current instance, the Bight of the First Section is arranged in or near the Second Section. It should be understood that the First Section is likely to remain moveable in relation to the Second Section such that a portion of a Standing End will transition to being a Bight then to the other Standing End as the portion of suture passes through the Second Section. In other words, when a suture portion is indicated to be a Standing End or a Bight as part of any description, it should be understood that the portion of suture being described is only in the particular location at that moment of assembly or use. For example, it is likely that one of the Standing Ends of a First Section will be shifted to a portion of the Bight.

The term “Bight” has been replaced at times herein, for the sake of clarity, with the term “Vertex”. As will become more evident during the following discussion, the term Vertex helps to more easily describe a transition in direction of a suture for the formation of an acute angle, for example.

Suture, as the term is used and described herein, includes braided (i.e., multi-filament) suture and monofilament suture as well as any other metallic or non-metallic filamentary or wire-like material suitable for performing the function of a suture. This material can include both absorbable and non-absorbable materials.

### First Embodiment

FIGS. 1-6 disclose a method of manufacturing a soft suture anchor from a single, continuous length of suture 1. Generally, the suture anchor of this first embodiment is created by arranging a suture in a predetermined manner designed to create a construct in which a portion of a First Section is slidably contained within a portion of a Second Section, the First Section and the Second Section being initially arranged in relation to one another as a single length of suture. More specifically, the Second Section is arranged in a manner around or surrounding a portion of the First Section with the two Sections being connected as part of a single length of suture. Once the desired arrangement of the Second Section about the First Section is completed, the construct may be cut, thereby separating the First Section and the Second Section, a portion of the First Section being slidably contained within the Second Section.

While not necessary for understanding the manufacturing steps of this first embodiment, it may be helpful to understand that a finished suture anchor made in accordance with this first embodiment is to be arranged in or on a driver in such a way as to enable the Second Section and a portion of the First Section to be advanced into or through a pre-formed hole. For and during advancement, the diameter of the Second Section is minimized to be equal to or less than the hole diameter. Upon deployment, the suture anchor diameter is maximized to be greater than the hole diameter to create a retention capacity. Deployment could occur, for example, in cancellous bone surrounding a hole formed in cortical and cancellous bone. The diameter of the deployed anchor would be positioned under the cortical layer. Deployment could also occur on a surface of bony or soft tissue after the Second Section is passed through a hole in the bony or soft tissue.

FIG. 1 shows the first step of forming the desired suture anchor in accordance with an embodiment of the present invention. A length of suture material 1 is arranged about a center pin 2 to form a first Vertex 15 between a first Standing End 10 and a second Standing End 20. Further shown in FIG. 1, the second Standing End 20 is to be arranged around a right side pin 3 to form a second Vertex 25 between the second Standing End and a Second Section 30 of suture. A side pin 4 may be used to hold first Standing End 10 in a position for further assembly. Additional pins shown in these and other figures are being utilized only to hold the suture 1 for clarity purposes.

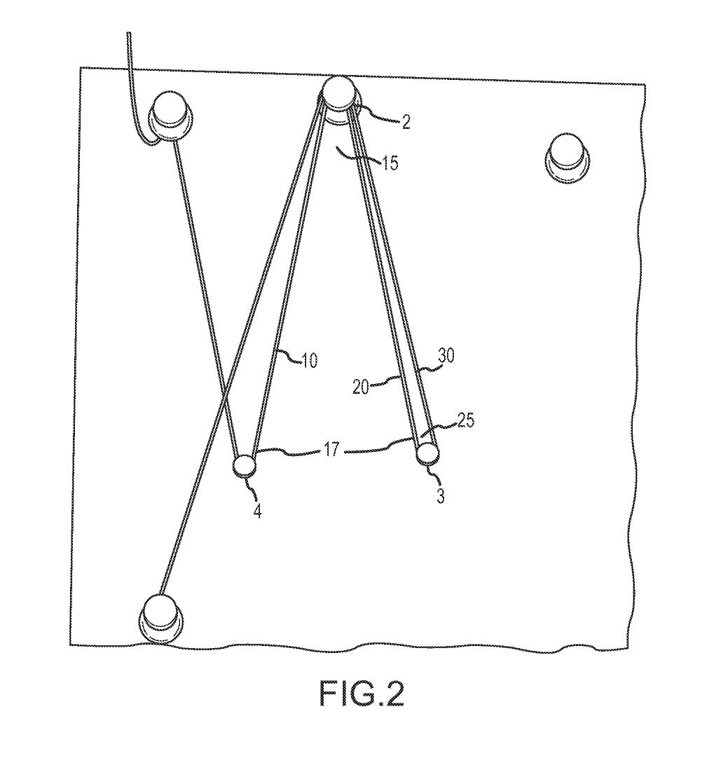

FIG. 2 shows a subsequent step where the Second Section 30 is arranged about the right side pin 3 to extend along the second Standing End 20 and is arranged about the center pin 2 to extend at least a partial distance along the first Standing End 10.

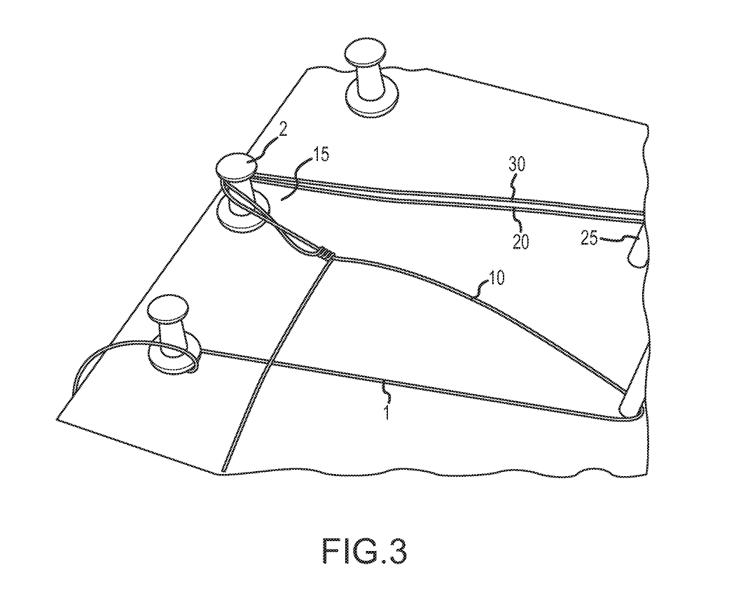

FIG. 3 shows a subsequent step where the Second Section 30 is wrapped a plurality (e.g., three in the present instance) of times in a counter-clockwise manner (as viewed from the center pin 2) about the first Standing End 10 and helically pitched away from the center pin 2. It has been discovered that the number of wraps has a significant effect of the retention capacity of an installed anchor. To a point, the number of wraps helps to increase a deployed diameter of the resulting Second Section 30. Too many wraps may, however, reduce the retention capacity and the Second Section 30 may fail to deploy properly.

Further in relation to the step shown in FIG. 3, the wraps of the Second Section 30 about the first Standing End 10 may occur at any reasonable distance from the center pin 2. The wraps of the Second Section may be slid closer to the center pin 2 and the vertex 15 after the arrangement of the suture is complete.

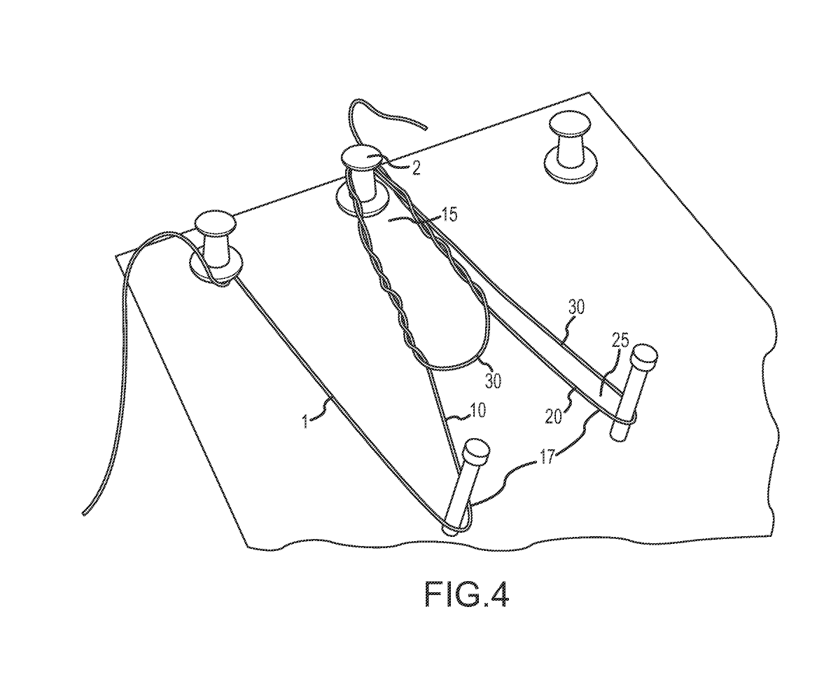

Lastly in relation to the step shown in FIG. 3, note that the wrapping of the Second Section 30 about the first Standing End 10 may occur in the clockwise direction as viewed from the center pin 2. As will become more apparent after reading the description of the step shown in FIG. 4, the wrap is to be accomplished to result in the passing of the Second Section 30 from the last wrap (around the first Standing End 10) to the second Standing End 20 either above (toward a viewing person of FIG. 3) both the first Standing End 10 and the second Standing End 20 or below (away from a viewing person of FIG. 3) both the first Standing End 10 and the second Standing End 20.

FIG. 4 shows a step including passing a portion of the Second Section 30 across an acute angle 17 formed by the first Standing End 10 and the second Standing End 20 such that the Second Section 30 is extending above (closer to a person viewing FIG. 4) both the first Standing End 10 and the second Standing end 20. Afterward, the Second Section is wrapped a plurality of times about the second Standing End 20 in a counter-clockwise manner, as viewed from the center pin 2 and helically pitched toward the center pin 2.

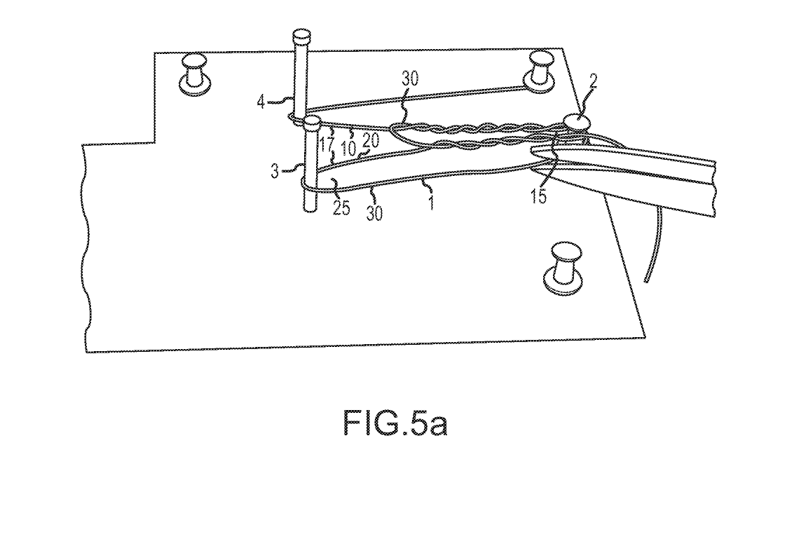

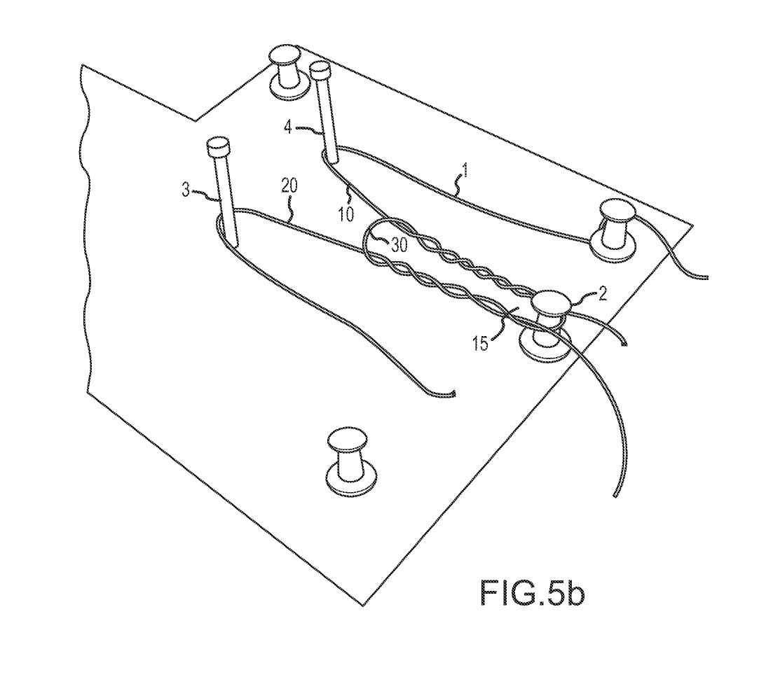

At this point it may help to remember that a primary goal of the all of the embodiments is to have a small diameter for the purposed of passing into or through a hole, while having an ability to increase laterally in diameter as part of deployment. Using FIGS. 4, 5a and 5b as an example, the winding on the Second Section 30 about the First Section is loose in terms of length from the center pin 2, which is used in this example only as a reference location. Accordingly, if/when the Standing Ends 10, 20 are drawn toward one another the overall assembly will have a relatively narrow profile, only slightly larger than the Standing Ends 10, 20 themselves. When deployed, however, the strands of the Second Section become drawn together, possibly creating a column of sorts, to result in a largest possible lateral diameter increase.

Again as described above, the number if wraps may vary, but it is preferred that the number of wraps about the first Standing End 10 be the same as or similar to the number of wraps about the second Standing End 20. Further, the direction of wrapping may be reversed if the wrapping about the first Standing End is also reversed. Lastly, the distance of the wrap from the center pin 2 is not a concern for the reasons discussed above.

It is envisaged that the wraps of the Second Section 30 about the first Standing End 10 and the second Standing End 20 may be reversed in term of their direction of helical pitch toward or away from the center pin 2. In the present FIG. 4, the wraps formed by the Second Section 30 on Standing Ends, 10, extend helically toward the center pin 2 from a viewpoint of Vertex 15, but it is envisaged that they could extend helically away from the center pin 2. Even so, it is believed that both must extend helically in the same direction (i.e., toward or away from) in relation to the center pin 2.

FIG. 5a shows a subsequent step of separating the Second Section 30 from the second Standing End 20 through the use of scissors or the like. It should be understood that the precise location of separation is not fixed and may be adjusted to result in an appropriate length for the Second Section 30. The resulting length of the second Standing End 20 may be adjusted by drawing the suture 1 through the Second Section 30.

FIG. 5b shows the result after the cut has been made.

Using FIG. 5b as a reference, it is contemplated that the number of “free” ends may become confusing, especially in the confines experienced during surgery. This issue is of special concern in light of these “all suture” anchors because of the additional suture material present. For example, an anchor of any of the five embodiments, or variations thereof, may be compromised if a surgeon were to inadvertently mistake an end of the Second Section 30 for one of the Standing Ends 10, 20. Accordingly, it is envisioned that there be some form of color change between the Second Section 30 and the Standing Ends 10, 20. This could be accomplished by having set lengths of particular colors or dying the filaments in the first or other embodiments where one length of suture is used to create the First Section 10, 15, 20 and the Second Section 30. Otherwise, as in the second embodiment, a suture of one color may be used for the First Section, 10, 15, 20 and a suture length of another color may be used for the Second Section 30.

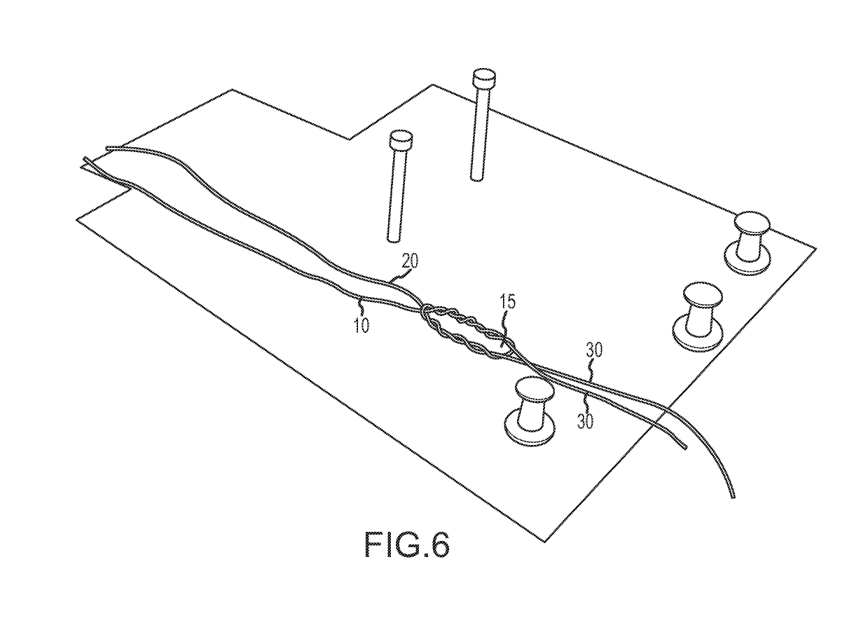

FIG. 6 shows the First Section (i.e. first Standing End 10, Vertex 15, and second Standing End 20) and the Second Section 30 of this particular construct after it has been removed from the forming pins.

The overall length of the First Section (first Standing End 10, Vertex 15, and second Standing End 20) may be made long enough to extend outside a recipient's body during arthroscopic procedures. The overall length of the Second Section 30 may be shortened to complete the manufacture of this anchor of the first embodiment, so as not to interfere with the deployment of anchor in a bone hole (not shown).

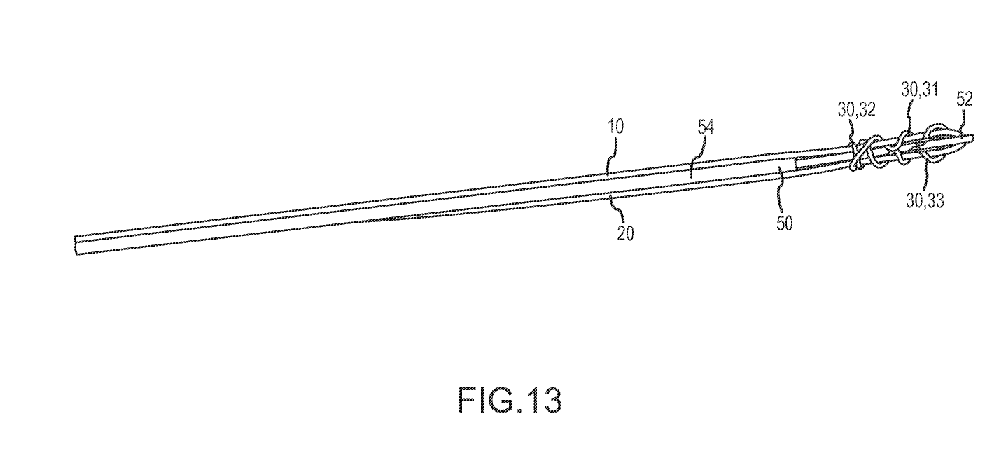

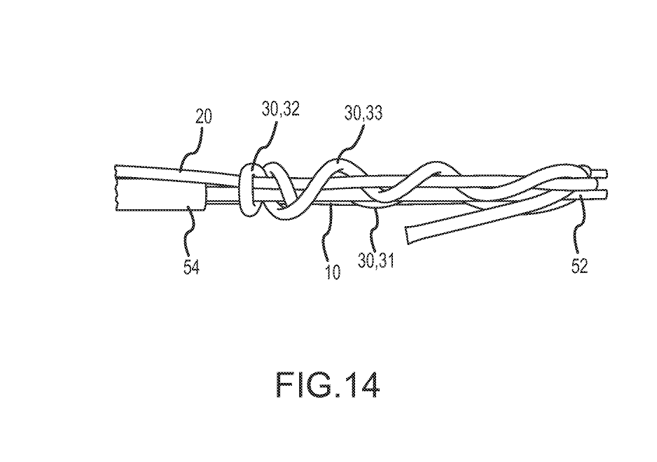

It will be understood that the resultant anchor could be formed as shown above without any driver or other body being utilized during the formation. The First Section (first Standing End 10, Vertex 15, and second Standing End 20) and the Second Section 30 may be placed within an elongated tube or sheath (not shown) for delivery to a surgical work site during an arthroscopic surgical procedure. Alternatively, a driver 50 (such as shown in FIGS. 13 and 14) (also referred to as a pusher) having a diameter on the order of 1 to 2 mm could be aligned longitudinally with the wrapping of the Second Section 30 during the formation process, thus resulting in an anchor being formed around driver 50.

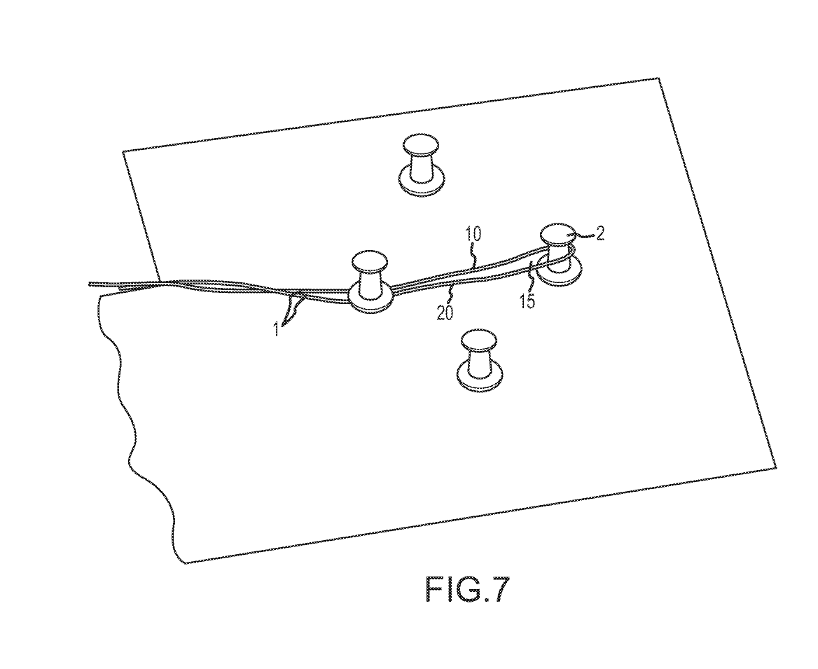

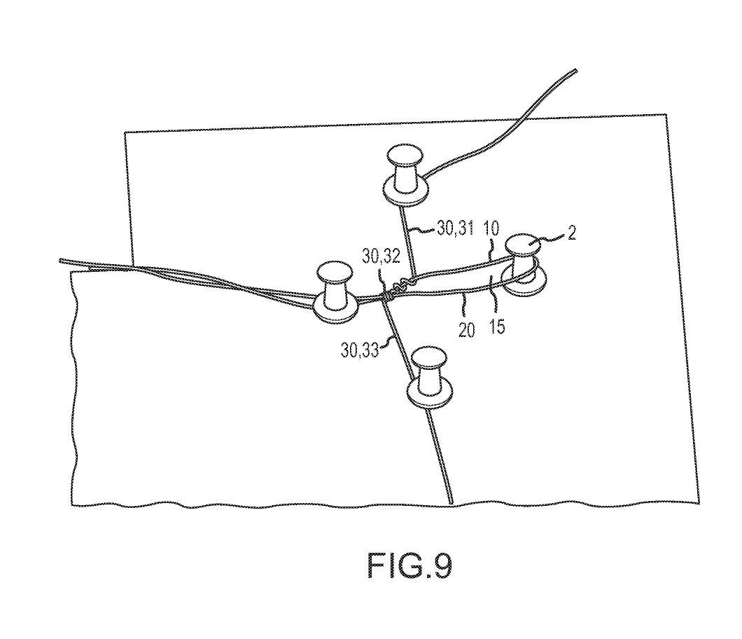

It may be helpful to note at this point that a resulting anchor shown in FIGS. 13 and 14 attached to driver 50 may be a variation of embodiment one (FIGS. 1-6) in addition to being the second embodiment (FIGS. 7-14). As shown in FIG. 4 and as described in relation to FIG. 4, the Second Section 30 is passed across the acute angle 17 prior to being wrapped about the second Standing End 20. A variation of this embodiment would be to further wrap, at least once, the Second Section 30 around (i.e., circumscribing) the second Standing End 20 and the first Standing End 10 (See FIG. 9, for example) before proceeding to wrap the Second Section 30 about only the second Standing End 20. This additional, circumscribing wrap allows the anchor to be retained against the driver 50 in two locations, at the Vertex 15 and the additional, circumscribing wrap (See reference 32 in FIG. 14, for example).

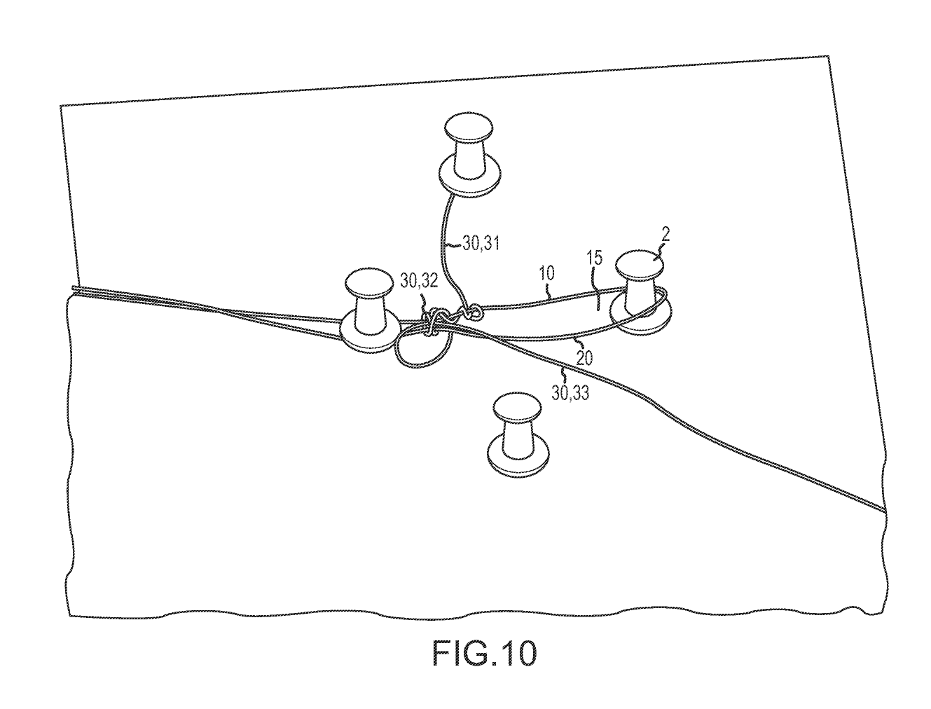

If the direction of this additional wrap 32 (FIG. 10) is away from the center pin 2, the Second Section 30 may be passed within the additional, circumscribing wrap 32 (FIG. 10) before being wrapped around only the second Standing End 20 (See FIG. 10, for example).

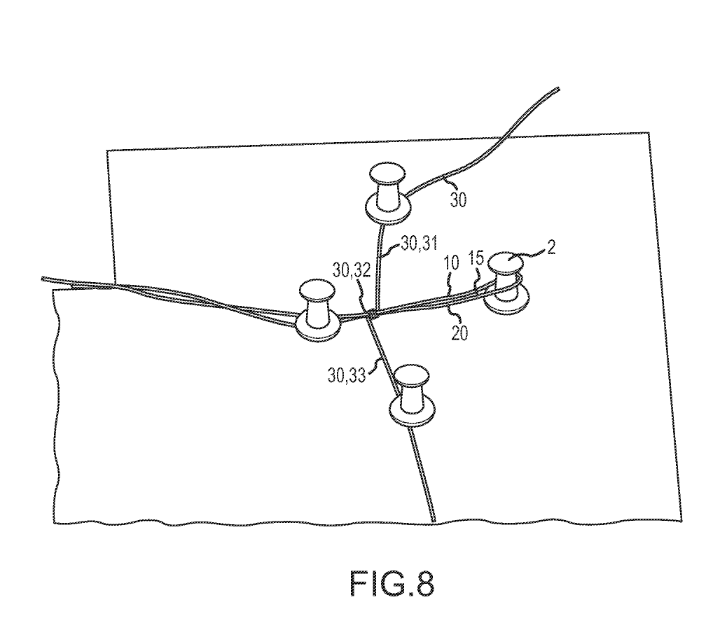

While these variations of the first embodiment are not explicitly shown in FIGS. 1-6 of the first embodiment, they are shown in FIGS. 8 and 10 of the second embodiment. Provided with these descriptions it should be clear that these variations are easily understood and incorporated without additional figures.

### Second Embodiment

FIGS. 7-14 disclose a method of manufacturing a variation of a soft suture anchor in accordance with a second embodiment. Portions of the method disclosed in FIGS. 7-14 are applicable to and representative of variations to the manufacturing method and anchor of the first embodiment.

Please note that because of the close relationship between the first and second embodiments, the names and reference numbers will be similar. It may be helpful to note that the method of the second embodiment may be used in place of the method of the first embodiment if there is a need or benefit to using a separate type (i.e., size, braid, material, color, etc.) of suture for the Second Section 30 than used in the First Section (i.e., first Standing End 10, Vertex 15, second Standing End 20).

FIG. 7 shows the first step of forming a suture anchor in accordance with an embodiment of the present invention. A length of suture material is arranged about a center pin 2 to form a first Vertex 15 between a first Standing End 10 and a second Standing End 20. The additional pins are being utilized only to hold the suture 1 for clarity purposes.

FIG. 8 shows a subsequent step where a Second Section (which could be a portion of suture 1 as in the first embodiment) is wrapped to circumscribe the first Standing End 10 and the second Standing End 20, the wrap being referenced as 32. For ease of identification the far side of the Second Section 30 will be referenced as the left Second Section 31 while the near side of the Second Section 30 will be referenced as the right Second Section 33.

Further in relation to the step shown in FIG. 8, the wraps 32 of the Second Section 30 may occur at any reasonable distance from the center pin 2. The wraps 32 may be slid closer to the center pin 2 and the vertex 15 after subsequent step or steps are complete.

FIG. 9 shows a subsequent step where the left Second Section 31 is wrapped a plurality (e.g., three in the present instance) of times in a counter-clockwise manner (helically pitching toward the center pin 2 and as viewed from the center pin 2) about the first Standing End 10. It has been discovered that the number of wraps has a significant effect of the retention capacity of an installed anchor. To a point, the number of wraps helps to increase a deployed diameter of the resulting Second Section 30. Too many wraps may, however, reduce the retention capacity and the Second Section 30 may fail to deploy properly.

Lastly in relation to the step shown in FIG. 9, note that the wrapping of the left Second Section 31 about the first Standing End 10 may occur in the clockwise direction as viewed from the center pin 2 if the wraps 32 are performed opposite from as shown.

FIG. 10 shows a subsequent step where the right Second Section 33 is passed through the wrap 32 prior to wrapping the right Second Section 33 around the second Standing End 20. This arrangement helps to stabilize the wraps 32 such that the wraps 32, and the remainder of the Second Section 30 are not prone to spill (i.e., unravel) based on the direction of wrap 32 in relation to other wraps of the Second Section 30.

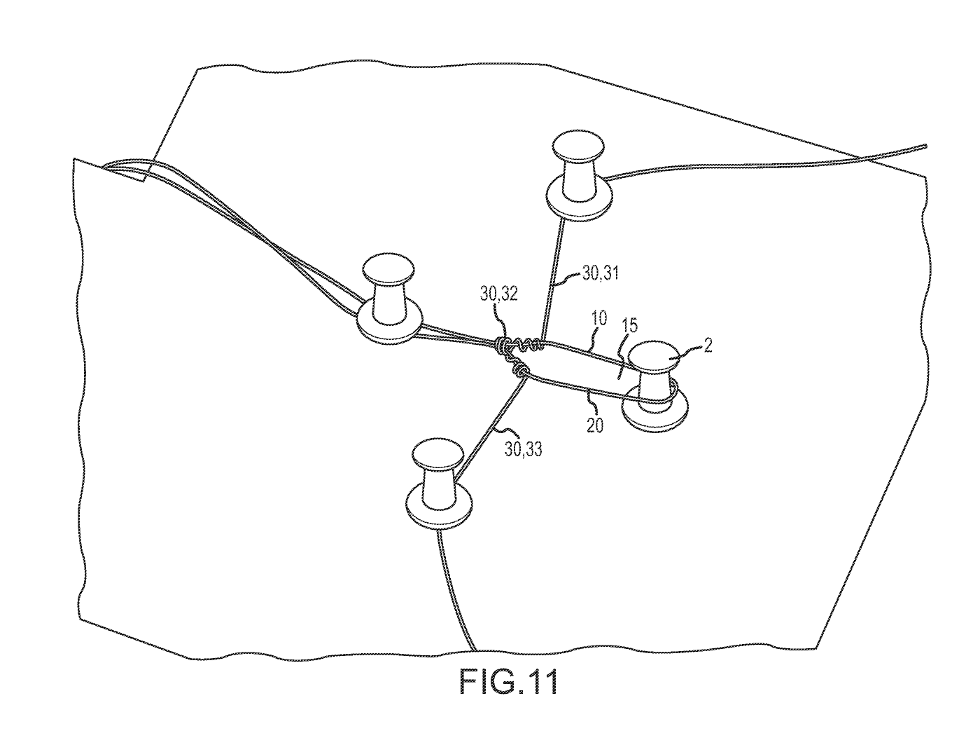

FIG. 11 shows a subsequent step where the right Second Section 33 is wrapped in a clockwise manner (as viewed from the center pin) a plurality of times (shown in FIG. 11 as three) about the second Standing End 20 and helically pitching toward the center pin 2. Again as described above, the number of wraps may vary, but it is preferred that the number of wraps about the first Standing End 10 be the same as or similar to the number of wraps about the second Standing End 20. Further, the direction of wrapping may be reversed if the wrapping about the first Standing End 10 is also revered.

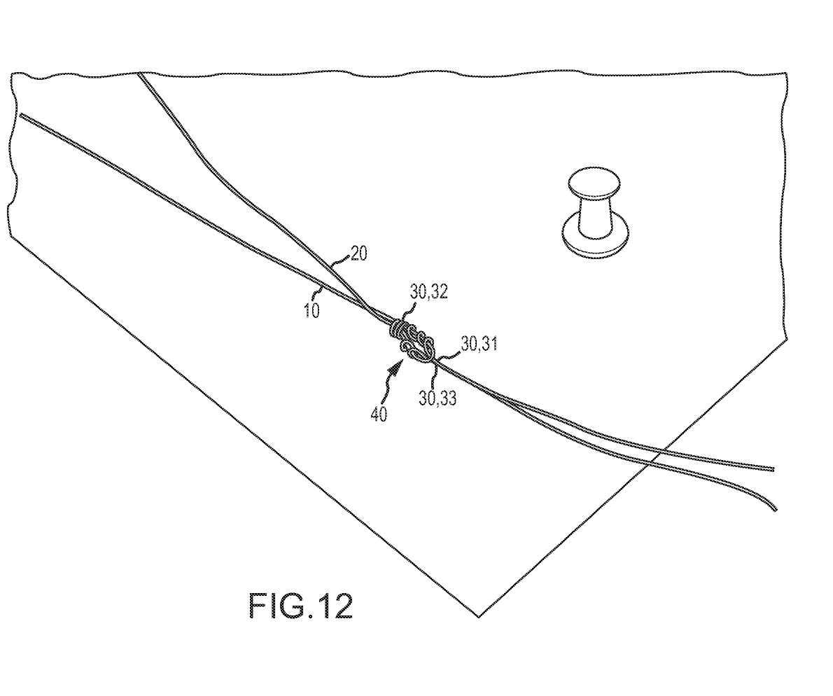

FIG. 12 shows a subsequent step where the left Second Section 31 is passed under (away from a viewer of FIG. 12) the Vertex 15 along with the right Second Section 33. In light of the counter rotation of the wrapping of the left Second Section 31 in relation to the wrapping of the right Second Section 33, this passage of suture under the Vertex 15 helps to prevent spilling of the wraps. If all of the wraps of the Second Section 30 were to be reversed, the left Second Section 31 and the right Second Section 33 may be passed above the Vertex 15.

It may be helpful to understand that the Second Section 30 in FIG. 12 is shown as being quite long. In other words, Section 30 will be shortened from its length in FIG. 12 before completion of the anchor.

It should be understood that the anchor could be formed as shown above without any driver or other body being utilized during the formation, as shown. Alternatively, driver (also referred to as a pusher) having a diameter on the order of 1 to 2 mm and shown in FIGS. 13 and 14 could be in place during the method steps shown in FIGS. 7-12, thus resulting in the anchor being formed around driver 50.

As shown in FIG. 13, and enlarged in FIG. 14, such a construct could have anchor formed about the distal end of driver 50 and be mounted thereon at two points. One point could be a forked end 52 (or other means) may pass through the wrap(s) 32 of the Second Section 30 and further to would engage Vertex 15 to enable driver 50 to push the suture anchor distally. The other point could be within wrap(s) 32 of the Second Section 30 as well as shaft 54 of driver 50. Shaft 54 could be cylindrical, rectangular or any other cross-section and forked end 52 could be replaced by a point (not shown) adapted to pierce (and push) the distal end of Vertex 15. FIG. 14 is a view of FIG. 13 more clearly showing the tines of forked end 52. For stability, shaft 54 could be slidingly received within an optional sheath (not shown) with suture limbs situated outside the sheath.

### Third Embodiment

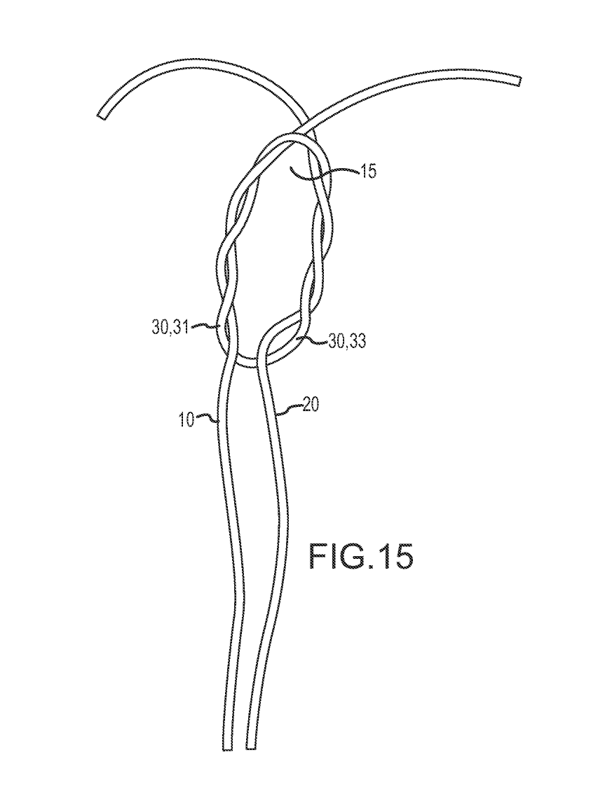

As shown in FIG. 15, a First Section (a first Standing End 10, a Vertex 15, and a second Standing End 20) are likely separate from a Second Section 30 during forming. In relation to the second embodiment, instead of wraps 32 (FIGS. 8-14), the first Standing End 10 and the second Standing End 20 each pierce through Second Section 30 near a midpoint of the Second Section 30. In relation to the first embodiment, the piercing of the Second Section 30 would take place where the Second Section 30 is passed across the acute angle 17 to the second Standing End 20.

While this “piercing” of the Second Section 30 with the each of the Standing Ends 10, 20, this feature may be used in other embodiments, in this or some other form, for the purpose of maintaining equal lengths of Second Section 30 on either side of the Standing Ends, for example.

### Fourth Embodiment

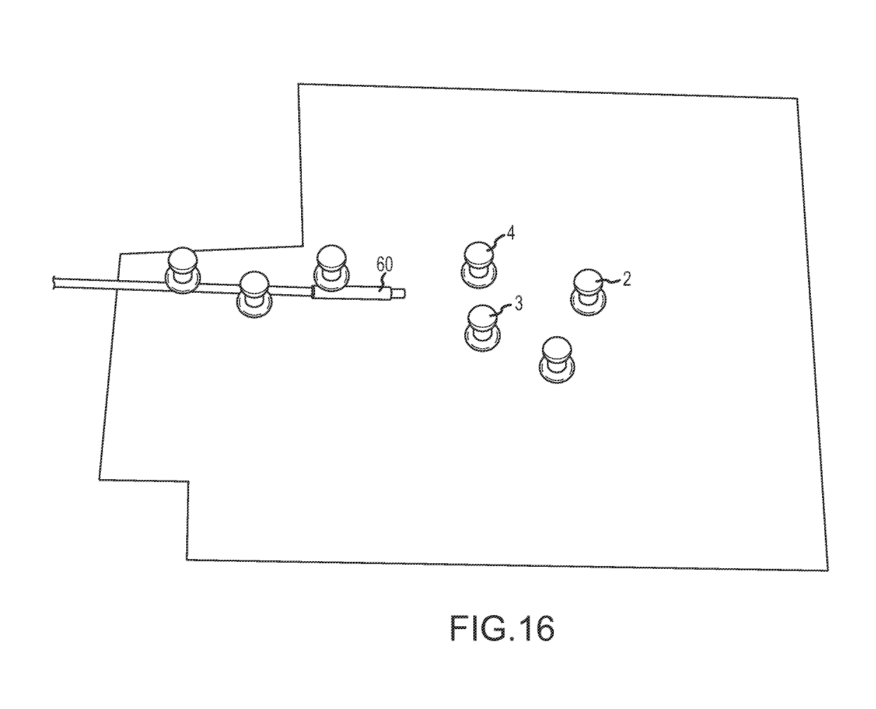

Referring now to FIGS. 16-23, a fourth embodiment is shown that may be considered a variation of the second embodiment in terms of method of manufacture and resulting anchor, and may be considered a variation of the first embodiment is terms of method of manufacture and resulting anchor. Note that the insertion sleeve 60 referenced in each of FIGS. 16-23 is relevant and applicable to each of the five embodiments discussed in the present application.

Please note that because of the close relationship between the first, second, and third embodiments, the names and reference numbers will be similar in this discussion of the fourth embodiment. It may be helpful to note that the method of the second embodiment may be used in place of the variation referred to here as the fourth embodiment.

FIG. 16 shows the first step of forming a suture anchor in accordance with a fourth embodiment of the present invention. A sheath 60 is arranged in line with a center pin 2 along with a right side pin 3 and a left side pin 4 on adjacent sides between a distal end of the sheath 60 and the center pin 2.

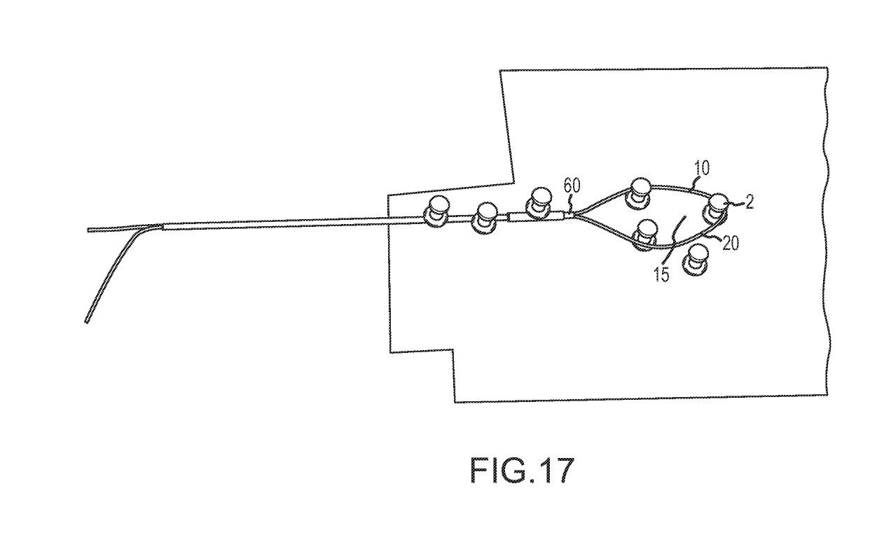

Referring now to FIG. 17, a length of suture material constituting a First Section (i.e., a first Standing End 10, a Vertex 15, and a second Standing End 20) is arranged about a center pin 2 to form a first Vertex 15 between the first Standing End 10 and the second Standing End 20. Each of the first Standing End 10 and the second Standing End 20 extend proximally from the center pin 2 circumscribe the right side pin 3 and the left side pin 4 before passing into a lumen (not referenced) within the sheath 60 to then extend proximally from a proximal end of the sheath 60.

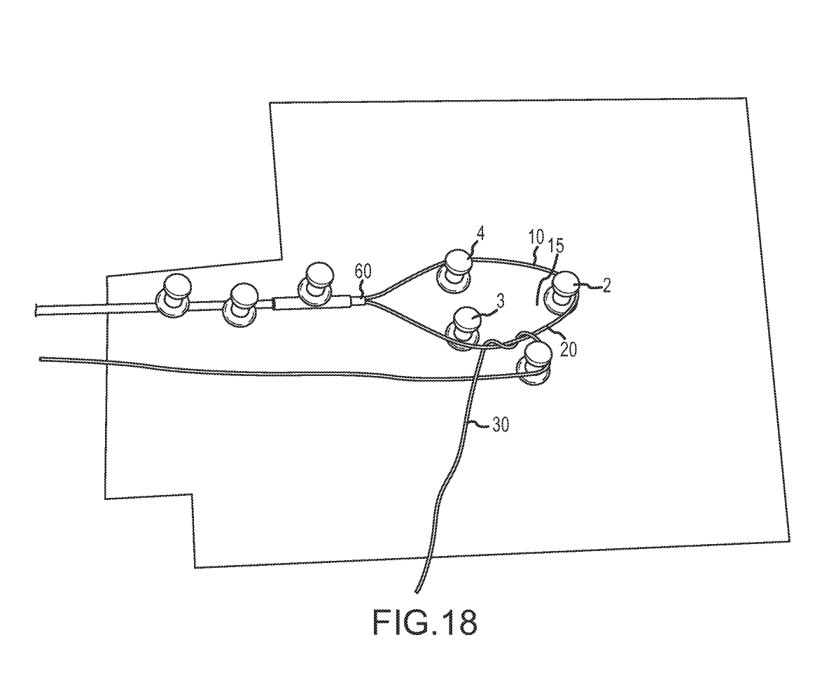

FIG. 18 shows a subsequent step where a Second Section 30 is wrapped about the second Standing end 20 to helically progress proximally in a clockwise manner, as viewed from the center pin 2.

FIG. 18 shows a subsequent step where a Second Section (which could be a portion of either the first Standing End 10 or the second Standing End) is wrapped a plurality (e.g., 2 in the present instance) of times in a clockwise manner (as viewed from the center pin 2) about the second Standing End 20 and helically pitched away from the center pin 2. It has been discovered that the number of wraps has a significant effect of the retention capacity of an installed anchor. To a point, the number of wraps helps to increase a deployed diameter of the resulting Second Section 30. Too many wraps may, however, reduce the retention capacity and the Second Section 30 may fail to deploy properly. IN this present embodiment, the Second Section 30 has fewer wraps (i.e. two instead of three in the first embodiment), but as will be seen in the following steps, the fourth embodiment includes two iterations of wraps of the Second Section 30 about each of the first Standing End 10 and the second Standing End 20.

Further in relation to the step shown in FIG. 18, the wraps of the Second Section 30 about the first Standing End 10 may occur at any reasonable distance from the center pin 2. The wraps of the Second Section may be slid closer to the center pin 2 and the Vertex 15 after the arrangement of the Second Section 30 is complete.

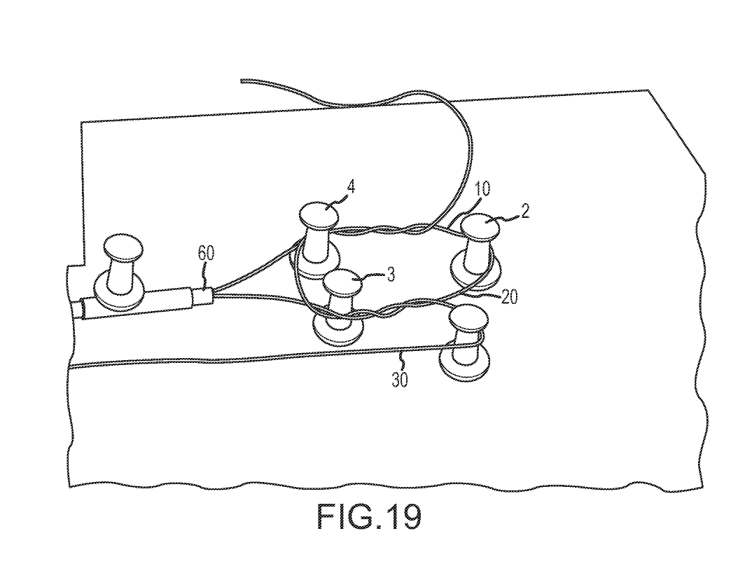

Lastly in relation to the step shown in FIG. 18, note that the wrapping of the Second Section 30 about the second Standing End 20 may occur in the counter-clockwise direction as viewed from the center pin 2. As will become more apparent after reading the description of step shown in FIG. 19, the wrap is to be accomplished to result in the passing of the Second Section 30 from the last wrap to the first Standing End 10 either above (toward a viewing person of FIG. 19) both the first Standing End 10 and the second Standing End 20 or below (away from a viewing person of FIG. 19) both the first Standing End 10 and the second Standing End 20.

FIG. 19 shows a step including passing a portion of the Second Section 30 across a distance between the first Standing End 10 and the second Standing End 20 such that the Second Section 30 is extending above (toward a person viewing FIG. 19) both the first Standing End 10 and the second Standing end 20. Afterward, the Second Section 30 is wrapped a plurality of times about the first Standing End 10 in a clockwise manner, as viewed from the center pin 2 and helically pitched toward the center pin 2.

Again as described above, the number if wraps may vary, but it is preferred that the number of wraps about the first Standing End 10 be the same as or similar to the number of wraps about the second Standing End 20. Further, the direction of wrapping may be reversed if the wrapping about the first Standing End is also revered. Lastly, the distance of the wrap from the center pin 2 is not a concern for the reasons discussed above.

It is envisaged that the wraps of the Second Section 30 about the first Standing End 10 and the second Standing End 20 may be reversed in term of their direction of helical pitch toward or away from the center pin 2. In the present FIG. 19, the wraps extend helically toward the center pin 2 from where the Second Section 30 passes from the second Standing End 20 to the first Standing End 10, but it is envisaged that they could extend helically proximally from the center pin 2. Even so, it is believed that both must extend helically in the same direction (i.e., toward or away from) in relation to the center pin 2.

Lastly in relation to FIG. 19 The Second Section 30 could be separated from the First Section as is accomplished in FIG. 5a discussed more fully above. However the present embodiment includes a second iteration, which is where the Second Section is wrapped and passed about the First Section a second time, whereas each of the first, second and third embodiments include only one iteration. This second iteration, which is shown in FIGS. 19-23, has been found to be beneficial in certain circumstances.

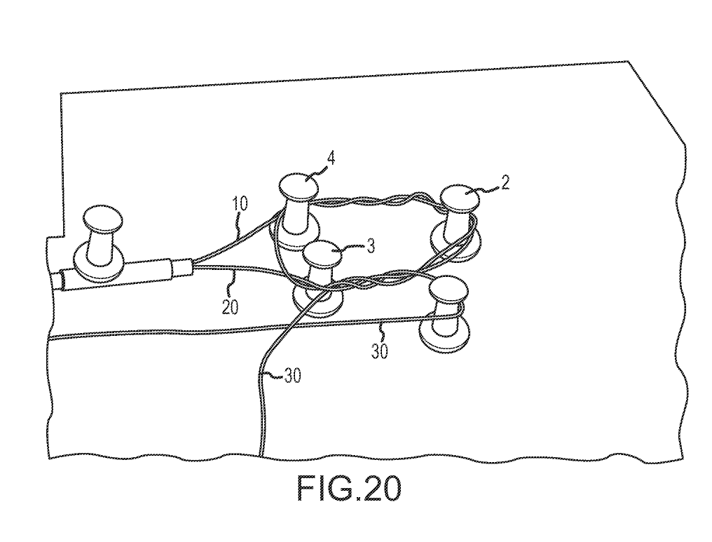

FIG. 20 shows a beginning of the second iteration, discussed above. In the present step, the Second Section is wrapped about the second Standing End 20 for a second iteration.

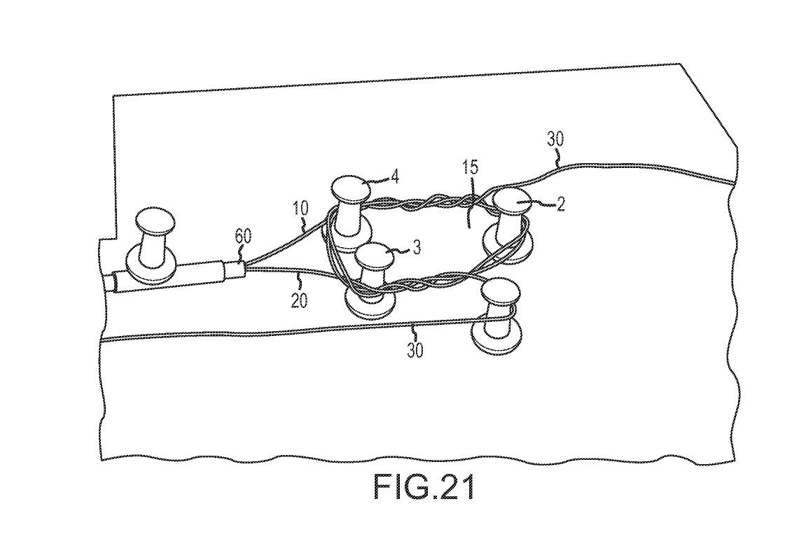

As shown in FIG. 21, the Second Section 30 is passed from the second Standing End 20 to the first Standing End 10 and then wrapped about the first Standing End 10.

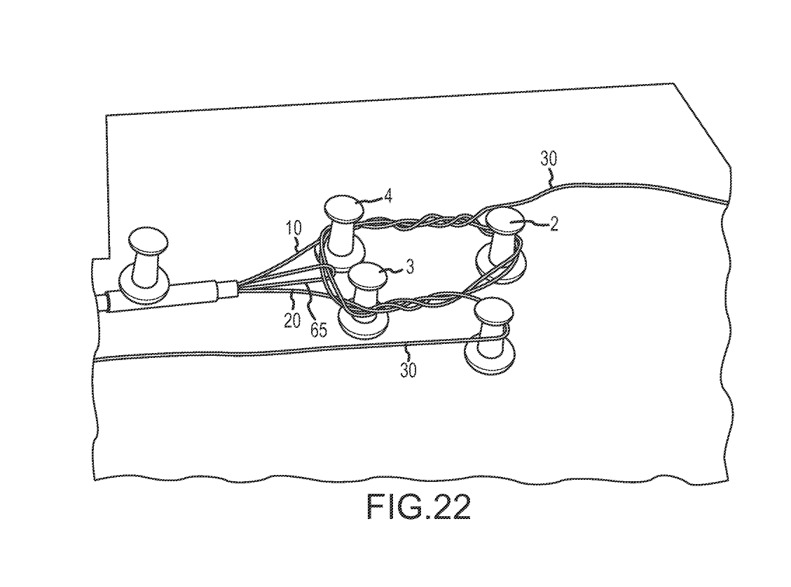

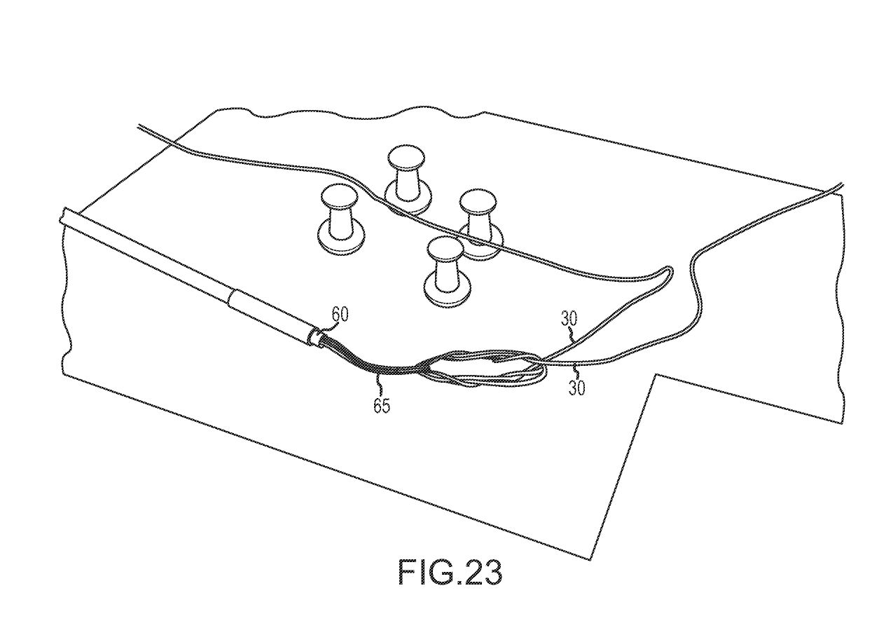

Referring now to FIG. 22, a retention loop 65 is passed from the sheath 65 and passed around portions of the Second Section 30 extending between the second Standing End 20 and the first Standing End 10. This retention loop may be used to pull the Second Section 30 within the sheath 60.

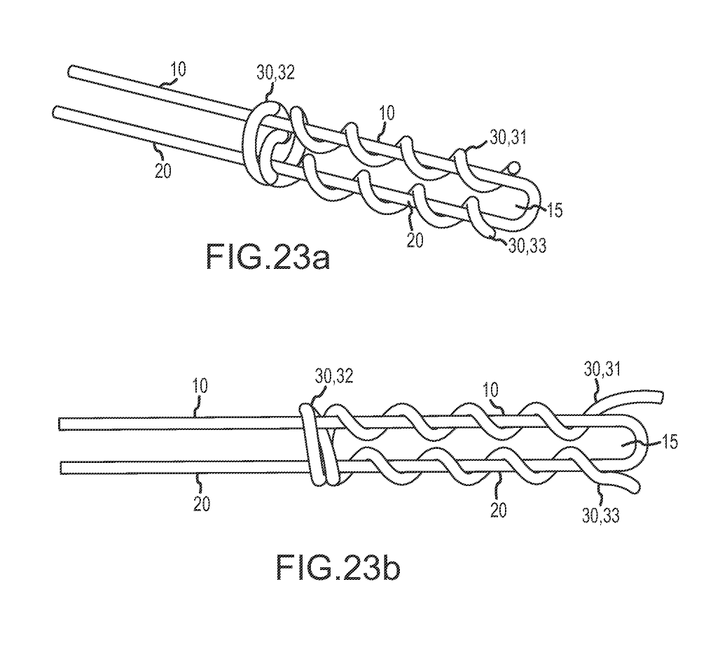

FIG. 23 shows a completed anchor remaining outside the sheath 60.

### Fifth Embodiment

FIGS. 23a and 23b shown a resultant suture anchor similar in many aspects to the embodiments discussed above. In the present embodiment, a Second Section 30 includes a wrap 32 that includes a second “pass” between a first Standing End 10 and a second Standing End 20. More specifically, after a last wrap of the left Second Section 31 about the first Standing End 10, the Second Section 30 is passed from the first Standing End 10 to the second Standing End 20 “under” both Ends 10, 20, and then passed between from the second Standing End 20 to the first Standing End 10 “over” both Ends 10, 20. Then the Second Section 30 is passed between the first Standing End 10 and the second Standing End 20 and between (i) the “under” passing and (ii) the “over” passing before being wrapped about the second Standing End 20. In other words, an end of the Second Section is passed between the first Standing End 10 and the second Standing End 20 at a location dividing at least one wrap 32 of the second section 30 into a distal portion of the wrap 32 and a proximal portion of the wrap 32, as shown in FIG. 15. It should be understood that there may be more than one wrap 32 and the benefit many be obtained by passing an end of the Second Section 30 between any one of the wraps 32.

This arrangement of wrap 32 allows for the wrapping of the Second Section 30 about the Standing Ends 10, 20 to occur helically in the same direction with one another.

It should be understood that the present description of this fifth embodiment could be reversed as a matter of choice or manufacturing.

Further, it should be noted that the specific number of wraps of the Second Section 30 about any portion of the Standing Ends 10, 20, separately 31, 33 or together 32, may be varied to accomplish a highest retention strength for a particular use.

All the processes described above may be automated and/or reversed to mimic the illustrated manual operations and may be scaled up or down to result in suture anchors having a pre-deployment diameter on the order to 1 to 2 mm. Other sizes may also be possible based on the designs and techniques discussed above.

1. A method of manufacturing a soft suture anchor, the method comprising:
providing a single length of suture;
bending the single length of suture to define a first vertex separating a first standing end of suture and a second standing end of suture, the first standing end and the second standing end being legs of an acute angle;
bending the second standing end to define a second vertex, suture extending beyond the second vertex being a second section of suture;
arranging the second section to extend along the second standing end, to extend about the first vertex and to extend a partial distance along the first standing end;
wrapping a first portion of the second section a plurality of times about the first standing end, the wrapping extending helically away from the first vertex;
upon a last wrap of the first portion of the second section about the first standing end, passing a second portion of the second section across the acute angle to the second standing end;
wrapping the second portion of the second section a plurality of times about the second standing end, the wrapping extending helically toward the first vertex; and
separating at least the portions of the second section wrapped around the first standing end and the second standing end from the first section.

2. The method of claim 1 wherein the step of passing the second section across the acute angle to the second standing end further comprises passing the second portion of the second section back as least back to the first standing end such that the second portion of the second section circumscribes the first standing end and the second standing end with at least one revolution.

3. The method of claim 2 further comprising passing a distal end of an insertion instrument though the at least one revolution of the second portion of the second section circumscribing the first standing end and the second standing end.

4. The method of claim 2 further comprising passing the second portion of the second section through the at least one revolution of the second portion of the second section circumscribing the first standing end and the second standing end.

5. The method of claim 2 further comprising passing the second portion of the second section between the first standing end and the second standing end at a location between (i) the second portion of the second section passing from the first standing end to the second standing end and (ii) the second portion of the second section passing from the second standing end to the first standing end.

7. The method of claim 1, wherein the single length of suture comprises at least one color change along its length, a first color provided along an expected length of said second portion.

8. The method of claim 1, wherein the single length of suture comprises at least two color changes along its length, a first color provided along an expected length of said second portion, a second color provided along an expected length of said first standing end, and a third color provided along an expected length of said second standing end.

9. A method of manufacturing a soft suture anchor, the method comprising:
providing a first section of suture and a second section of suture;
bending the first section to define a first vertex separating a first standing end and a second standing end, the first standing end and the second standing end being legs of an acute angle;
circumscribing the first standing end and the second standing end with at least one wrap of the second section;
wrapping a first end of the second section a plurality of times about the first standing end; and
wrapping a second end of the second section a plurality of times about the second standing end.

10. The method of claim 9 further comprising passing and end of the second section through said at least one wrap of the second section circumscribing the first standing end and the second standing end.

11. The method of claim 9 further comprising: passing a distal end of an insertion instrument through said at least on wrap of the second section circumscribing the first standing end and the second standing end; and engaging said first vertex of said first section.

12. The method of claim 9 further comprising passing an end of the second section between the first standing end and the second standing end at a location dividing said at least one wrap of the second section into a distal portion and a proximal portion.

13. A method of manufacturing a soft suture anchor, the method comprising:
providing a first section of suture having a first standing end and a second standing end separated from one another by a first vertex;
providing a second section of suture having a first free end and a second free end separated from one another by a second vertex;
orienting said first section and said section in a common plane with said first vertex and said second vertex facing one another;
wrapping said first standing end and said first free end a plurality of times about one another;
wrapping said second standing end and said second free end a plurality of times about one another; and
piercing said second vertex with each of said first standing end and said second standing end.

14. A soft suture anchor comprising:
a first section of suture bent to define a first vertex separating a first standing end and a second standing end, the first standing end and the second standing end being legs of an acute angle;
a second section of suture circumscribing the first standing end and the second standing end with at least one wrap;
a first end of said second section being wrapped a plurality of times about the first standing end; and
a second end of the second section being wrapped a plurality of times about the second standing end.

15. The soft suture anchor of claim 14 further comprising a portion of the second section extending through said at least one wrap of the second section circumscribing the first standing end and the second standing end.

16. The soft suture anchor of claim 14 further comprising: a distal end of an insertion instrument extending through said at least on wrap of the second section circumscribing the first standing end and the second standing end; and engaging said first vertex of said first section.

17. The soft suture anchor of claim 14 further comprising a portion of the second section extending between the first standing end and the second standing end at a location dividing said at least one wrap of the second section into a distal portion and a proximal portion.

18. The soft suture anchor of claim 14, wherein in a first state the second portion has a relatively narrow transverse diameter and in a second, deployed state, the second section has a relatively larger transverse diameter.

19. A soft suture anchor comprising:
a first section of suture having a first standing end and a second standing end separated from one another by a first vertex;
a second section of suture having a first free end and a second free end separated from one another by a second vertex, said first section and said section being oriented in a common plane with said first vertex and said second vertex facing one another;
said first standing end and said first free end being wrapped a plurality of times about one another;
said second standing end and said second free end being wrapped a plurality of times about one another; and
said second vertex being pierced with each of said first standing end and said second standing end.

20. The soft suture anchor of claim 19, wherein in a first state the second portion has a relatively narrow transverse diameter and in a second, deployed state, the second section has a relatively larger transverse diameter.
